(19) United States
(12) Patent Application Publication
(10) Pub. No.: US 2025/0222251 A1
(43) Pub. Date: Jul. 10, 2025

(54) IMPLANTABLE STIMULATION ARRANGEMENT STRUCTURES

(71) Applicant: Cochlear Limited, Macquarie University, NSW (AU)

(72) Inventors: Peter Gibson, South Coogee, NSW (AU); Shahram Manouchehri, Pemulwuy, NSW (AU); Nicholas Charles Kendall Pawsey, North Ryde, NSW (AU)

(21) Appl. No.: 18/728,579

(22) PCT Filed: Jan. 11, 2023
(86) PCT No.: PCT/IB2023/050249

§ 371 Date: Jul. 12, 2024

Related U.S. Application Data
(60) Provisional application No. 63/300,771, filed on Jan. 19, 2022.

(51) Int. Cl. A61N 1/05

(52) U.S. Cl. CPC A61N 1/0541

(57) ABSTRACT

Presented herein are techniques for mechanically decoupling (isolating) a stimulation assembly from an associated lead via pre-formed (e.g., pre-molded) discontinuity that connects a lead to an elongate stimulation assembly in a manner that substantially minimizes the transfer of torsional, linear, and/or angular forces from the lead to the stimulation assembly.

### BACKGROUND

### Field of the Invention

The present invention relates generally to implantable stimulation arrangements.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, an implantable medical device is provided. The implantable medical device comprises: a stimulator unit configured to be implanted in a recipient; an elongate stimulation assembly configured to be implanted in the recipient, wherein the elongate stimulation assembly includes at least one electrode; a lead comprising a resiliently flexible body and at least one wire disposed in the resiliently flexible body and electrically connecting the at least one electrode to the stimulator unit; and a decoupling structure physically connecting a proximal end of the elongate stimulation assembly to a distal end of the lead, wherein the decoupling structure mechanically decouples the elongate stimulation assembly from the lead.

In another aspect, an implantable stimulation arrangement is provided. The implantable stimulation arrangement comprises: an elongate stimulation assembly comprising an intra-cochlear region configured to be implanted in a cochlea of a recipient and an extra-cochlear region; an elongate lead; and an angular discontinuity attaching a proximal end of the extra-cochlear region to a distal end of the extra-cochlear region at a predetermined angle in a range of approximately 45 degrees to approximately 120 degrees.

In another aspect, an implantable stimulation arrangement is provided. The implantable stimulation arrangement comprises: an elongate stimulation assembly configured to be implanted in a recipient, wherein the elongate stimulation assembly comprises a plurality of electrodes; a lead; and an angular discontinuity disposed between a proximal end of the elongate stimulation assembly and a distal end of the lead, wherein the angular discontinuity comprises a pre-formed bend and an extension region that is non-parallel to an axis of a straight portion of a distal section of the elongate stimulation assembly within a body chamber of the recipient.

In another aspect, an implantable stimulation arrangement is provided. The implantable stimulation arrangement comprises: an elongate stimulation assembly configured to be implanted in a recipient; and a lead having a distal end connected to a proximal end of the elongate stimulation assembly, wherein, in an unstressed state, at least a distal end of lead is oriented substantially orthogonal to an axis of a straight portion of a distal section of the elongate stimulation assembly within a body chamber.

In another aspect, aspect, an implantable medical device is provided. The implantable medical device comprises: a stimulator unit configured to be implanted in a recipient; an elongate stimulation assembly configured to be implanted in the recipient, wherein the elongate stimulation assembly includes at least one electrode; a lead comprising a resiliently flexible body and at least one wire disposed in the resiliently flexible body and electrically connecting the at least one electrode to the stimulator unit; and a decoupling structure physically connecting a proximal end of the elongate stimulation assembly to a distal end of the lead, wherein the decoupling structure mechanically decouples the elongate stimulation assembly from the lead, wherein the decoupling structure comprises a pre-formed bend and a proximal extension having a predetermined first length oriented at a predetermined angle relative to an axis of a straight portion of a distal section of the elongate stimulation assembly within a body chamber of the recipient, wherein the pre-formed bend comprises an angle in a range of approximately 45 degrees to approximately 120 degrees relative to an axis of a straight portion of a distal section of the elongate stimulation assembly within a body chamber of the recipient, and wherein the proximal end of the elongate stimulation assembly has a diameter, and wherein the predetermined first length is at least five times the diameter of the elongate stimulation assembly.

### BRIEF DESCRIPTION OF THE DRAWINGS

Embodiments of the present invention are described herein in conjunction with the accompanying drawings, in which:

FIG. 1A is a schematic diagram illustrating a cochlear implant system comprising a stimulation arrangement with a decoupling structure, in accordance with certain embodiments presented herein;

FIG. 1B is a side view of a recipient wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIG. 2 is a schematic diagram illustrating an implantable stimulation assembly subject to forces transferred from an associated lead;

FIG. 3A is a perspective view of the implantable component of the cochlear implant system of FIG. 1A;

FIG. 3B is a side view of the implantable component of the cochlear implant system of FIG. 1A;

FIG. 4A is a schematic diagram illustrating a stimulation arrangement having a decoupling structure, in accordance with certain embodiments presented herein;

FIG. 4B is a side view of a portion of the stimulation arrangement of FIG. 4A;

FIG. 4C is a schematic diagram illustrating one example of the stimulation arrangement of FIG. 4A in which the decoupling structure operates to minimize transfer of forces from a lead to a stimulation assembly, in accordance with certain embodiments presented herein;

FIG. 4D is a schematic diagram illustrating one example of the stimulation arrangement of FIG. 4A in which the decoupling structure operates to minimize transfer of forces from a lead to a stimulation assembly, in accordance with certain embodiments presented herein;

FIG. 4E is a schematic diagram illustrating one example of the stimulation arrangement of FIG. 4A in which the decoupling structure operates to minimize transfer of forces from a lead to a stimulation assembly, in accordance with certain embodiments presented herein;

FIG. 4F is a schematic diagram illustrating one example of the stimulation arrangement of FIG. 4A in which the decoupling structure operates to minimize transfer of forces from a lead to a stimulation assembly, in accordance with certain embodiments presented herein;

FIG. 5 is a schematic diagram illustrating a stimulation arrangement having a decoupling structure, in accordance with certain embodiments presented herein;

FIG. 6A is a side view of a decoupling structure, in accordance with certain embodiments presented herein;

FIG. 6B is a perspective view of the decoupling structure of FIG. 6A;

FIG. 7 is a schematic diagram illustrating a stimulation arrangement having a decoupling structure, in accordance with certain embodiments presented herein;

FIG. 8 is a schematic diagram illustrating another stimulation arrangement having a decoupling structure, in accordance with certain embodiments presented herein;

FIG. 9 is a schematic diagram illustrating another stimulation arrangement having a decoupling structure, in accordance with certain embodiments presented herein;

FIG. 10 is a schematic diagram illustrating another stimulation arrangement having a decoupling structure, in accordance with certain embodiments presented herein;

FIG. 11 is a schematic diagram illustrating another stimulation arrangement having a decoupling structure, in accordance with certain embodiments presented herein; and

FIG. 12 is a diagram illustrating a vestibular stimulator system comprising a stimulation arrangement with a decoupling structure, in accordance with certain embodiments presented herein.

### DETAILED DESCRIPTION

A growing number of implantable medical devices include a stimulation assembly that is configured to be implanted within a recipient. The stimulation assembly is typically configured to deliver stimulation signals (e.g., electrical stimulation signals) to the recipient. For example, cochlear implants typically include a stimulation assembly configured to be implanted within, and configured to deliver stimulation to, the cochlea (e.g., scala tympani) of a recipient.

The stimulation signals delivered via the stimulation assembly are typically generated by a stimulator unit that is implanted some distance away from the stimulation assembly. The stimulator unit is electrically connected to the stimulation assembly via one or more wires extending through an electrical lead (lead or lead region) that are used to provide the stimulation signals to the stimulation assembly.

In general, the implanted position of a stimulator unit and a stimulation assembly can vary with different surgical approaches, different recipients (e.g., different size heads), and/or for other reasons. As such, the final distance between a stimulator unit and a stimulation assembly and the relative final orientations of the stimulator unit and a stimulation assembly can vary. Accordingly, implantable leads are often intentionally manufactured so as to be sufficiently long and sufficiently flexible to accommodate different final distances and different orientations between the stimulator unit and the stimulation assembly.

In conventional arrangements, the leads are directly mechanically coupled with the stimulation assembly. That is, conventional leads are attached to the stimulation assembly in a manner that enables torsional, linear, and/or angular forces to be transferred from the lead to the stimulation assembly. That is, by virtue of the physical structure of the connection between a conventional lead and stimulation assembly, mechanical manipulation (e.g., twisting, coiling, pushing, pulling, etc.) of the lead is transferred to the stimulation assembly. This mechanical coupling, when combined with the extra length of a lead, can cause surgical complications. More specifically, the extra length of the lead, although helpful to accommodate different relative positioning between a stimulation assembly and stimulator unit, often requires the surgeon to “coil” the lead within the recipient. In conventional arrangements, because the lead has stiffness and mechanical properties of its own that resists the coiling (e.g., movement and/or twisting of the lead), the coiling process results in corresponding movement and/or twisting of the stimulation assembly from its intended position (i.e., via the mechanical coupling between the lead and the stimulation assembly).

For example, with an elongate stimulation assembly having an intra-cochlear region, movement and/or twisting of the associated lead in the mastoid cavity can cause movement and/or twisting of the intra-cochlear region of the stimulation assembly (e.g., torsion twists the intra-cochlear region and puts pressure on the inner ear structures; linear over-insertion either pushes the intra-cochlear region onto the inner ear structures with uncontrolled force or pushes a perimodiolar stimulation away from the modiolus; or combinations thereof). That is, the lead acts like a beam that can translate torsional, linear and/or angular forces from the lead in the area of the mastoid cavity outside the cochlea to the intra-cochlear region of the stimulation assembly. Transfer of the torsional, linear, and/or angular forces to the stimulation assembly can, in turn, result in damage to intra-cochlear structures that causes loss of hearing (and an immediate loss of cochlea microphonic signal) and/or displacement or over insertion of the stimulation assembly, causing sub-optimal electrode position and reduction in electric hearing performance.

Presented herein are techniques for mechanically decoupling (isolating) a stimulation assembly from an associated lead via a “mechanical decoupling structure” or simply “decoupling structure.” As used herein, a “decoupling structure” is a pre-formed (e.g., pre-molded) discontinuity that connects a lead to an elongate stimulation assembly in a manner that substantially minimizes the transfer of torsional, linear, and/or angular forces from the lead to the stimulation assembly (e.g., the decoupling structure can isolate the intra-cochlear region from both these forces). In certain embodiments, as described further below, the decoupling structure creates a mechanical weakness that minimizes the transfer of the torsional, linear, and/or angular forces from the lead to the stimulation assembly.

In certain embodiments, the decoupling structure is the form of an angular discontinuity. As used herein, an “angular discontinuity” includes a pre-formed/pre-biased bend disposed between a proximal end of a stimulation assembly and a distal end of an associated (connected) lead, where the pre-formed bend is combined with a proximal extension (extension region). The proximal extension extends a minimum proximal distance from the pre-formed bend at a predetermined angle (of the pre-formed bend) relative to relative to an axis of straight portion of a distal section of the elongate stimulation assembly within a body chamber of the recipient. That is, the proximal extension has a minimum length, is located on an opposite side of the bend from the stimulation assembly, and is angled (non-parallel to), relative to an axis of straight portion of a distal section of the elongate stimulation assembly within a body chamber of the recipient. The minimal length of the proximal extension is selected to mechanically decouple the lead from the stimulation assembly. As such, the angular discontinuity physically connects a distal end of the lead to a proximal end of the stimulation assembly, but yet minimizes the transfer of torsional, linear, and/or angular forces from the lead to the stimulation assembly. In certain embodiments, the proximal extension can comprise a distal region of the lead.

Merely for ease of description, the techniques presented herein are primarily described with reference to a specific implantable medical device system, namely a cochlear implant system. However, it is to be appreciated that the techniques presented herein may also be partially or fully implemented by other types of devices or systems with other types of environmental signals. For example, the techniques presented herein may be implemented by other hearing devices, personal sound amplification products (PSAPs), or hearing device systems that include one or more other types of hearing devices, such as hearing aids, middle ear auditory prostheses, bone conduction devices, direct acoustic stimulators, electro-acoustic prostheses, auditory brain stimulators, cochlear implants, combinations or variations thereof, etc. The techniques presented herein may also be implemented by dedicated tinnitus therapy devices and tinnitus therapy device systems. In further embodiments, the presented herein may also be implemented by, or used in conjunction with, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, wearable devices, etc.

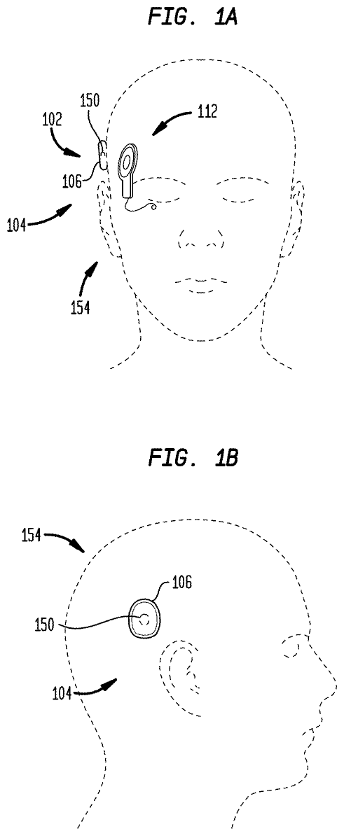

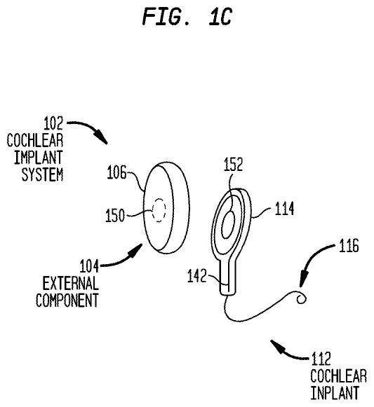

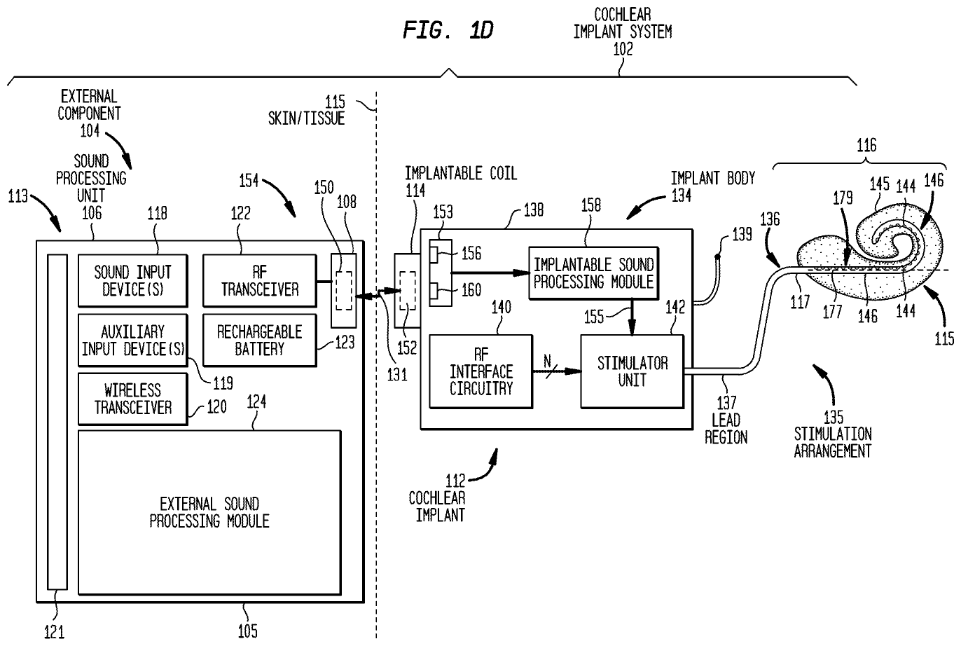

FIGS. 1A-1D illustrates an example cochlear implant system 102 with which certain embodiments of the techniques presented herein can be implemented. The cochlear implant system 102 comprises an external component 104 and an implantable component 112. In the examples of FIGS. 1A-1D, the implantable component is sometimes referred to as a “cochlear implant.” FIG. 1A illustrates the cochlear implant 112 implanted in the head 141 of a recipient, while FIG. 1B is a schematic drawing of the external component 104 worn on the head 141 of the recipient. FIG. 1C is another schematic view of the cochlear implant system 102, while FIG. 1D illustrates further details of the cochlear implant system 102. For ease of description, FIGS. 1A-1D will generally be described together.

As noted, cochlear implant system 102 includes an external component 104 that is configured to be directly or indirectly attached to the body of the recipient and an implantable component 112 configured to be implanted in the recipient. In the examples of FIGS. 1A-1D, the external component 104 comprises a sound processing unit 106, while the cochlear implant 112 includes an internal coil 114, an implant body 134, a lead 137, and an elongate stimulation assembly 116.

In the example of FIGS. 1A-1D, the sound processing unit 106 is an off-the-ear (OTE) sound processing unit, sometimes referred to herein as an OTE component, that is configured to send data and power to the implantable component 112. In general, an OTE sound processing unit is a component having a generally cylindrically shaped housing 105 and which is configured to be magnetically coupled to the recipient's head (e.g., includes an integrated external magnet 150 configured to be magnetically coupled to an implantable magnet 152 in the implantable component 112). The OTE sound processing unit 106 also includes an integrated external (headpiece) coil 108 that is configured to be inductively coupled to the implantable coil 114.

It is to be appreciated that the OTE sound processing unit 106 is merely illustrative of the external devices that could operate with implantable component 112. For example, in alternative examples, the external component may comprise a behind-the-ear (BTE) sound processing unit or a micro-BTE sound processing unit and a separate external. In general, a BTE sound processing unit comprises a housing that is shaped to be worn on the outer ear of the recipient and is connected to the separate external coil assembly via a cable, where the external coil assembly is configured to be magnetically and inductively coupled to the implantable coil 114. It is also to be appreciated that alternative external components could be located in the recipient's ear canal, worn on the body, etc.

As noted above, the cochlear implant system 102 includes the sound processing unit 106 and the cochlear implant 112. However, as described further below, the cochlear implant 112 can operate with the sound processing unit 106 stimulate the recipient or the cochlear implant 112 can operate independently from the sound processing unit 106, for at least a period, to stimulate the recipient. For example, the cochlear implant 112 can operate in a first general mode, sometimes referred to as an “external hearing mode,” in which the sound processing unit 106 captures sound signals which are then used as the basis for delivering stimulation signals to the recipient. The cochlear implant 112 can also operate in a second general mode, sometimes referred as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sound signals itself via implantable sound sensors and then uses those sound signals as the basis for delivering stimulation signals to the recipient. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 could also operate in alternative modes.

Referring first to the external hearing mode, FIGS. 1A-1D illustrate that the OTE sound processing unit 106 comprises one or more input devices 113 that are configured to receive input signals (e.g., sound or data signals). The one or more input devices 113 include one or more sound input devices 118 (e.g., one or more external microphones, audio input ports, telecoils, etc.), one or more auxiliary input devices 119 (e.g., audio ports, such as a Direct Audio Input (DAI), data ports, such as a Universal Serial Bus (USB) port, cable port, etc.), and a wireless transmitter/receiver (transceiver) 120. However, it is to be appreciated that one or more input devices 113 may include additional types of input devices and/or less input devices (e.g., the wireless short range radio transceiver 120 and/or one or more auxiliary input devices 119 could be omitted).

The OTE sound processing unit 106 also comprises the external coil 108, a charging coil 121, a closely-coupled transmitter/receiver (RF transceiver) 122, sometimes referred to as or radio-frequency (RF) transceiver 122, at least one rechargeable battery 123, and an external sound processing module 124. The external sound processing module 124 may comprise, for example, one or more processors and a memory device (memory) that includes sound processing logic. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

The implantable component 112 comprises an implant body (main module) 134 and a stimulation arrangement 135, all configured to be implanted under the skin/tissue (tissue) of the recipient. The implant body 134 generally comprises a hermetically-sealed housing 138 in which RF interface circuitry 140 and a stimulator unit 142 are disposed. The implant body 134 also includes the internal/implantable coil 114 that is generally external to the housing 138, but which is connected to the RF interface circuitry 140 via a hermetic feedthrough (not shown in FIG. 1D).

The stimulation arrangement 135 is described as comprising three (3) parts, namely the elongate stimulation assembly 116, the lead 137, and a decoupling structure 136 in the form of an angular discontinuity, which is referred to below as angular discontinuity 136. The stimulation assembly 116 generally comprises an intra-cochlear region 115 and an extra-cochlear or handle region 117.

In this example, the stimulation assembly 116 (the intra-cochlear region 115 and the extra-cochlear region 117) are mechanically decoupled from and the lead 137 by the angular discontinuity 136. As described further below, the angular discontinuity 136 is configured so as to minimize the transfer of torsional, linear, or angular forces placed on the lead 137 to the stimulation assembly 116. Further details regarding decoupling structures, such as angular discontinuity 136, are provided below.

As shown in FIG. 1D, the stimulation assembly 116 comprising a carrier member (e.g., resiliently flexible body formed, for example, from silicone) 139 that is configured to be at least partially implanted in the recipient's cochlea 145. Disposed in/on the carrier member 139 is a plurality of longitudinally spaced intra-cochlear electrical stimulating contacts (electrodes) 144 that collectively form a contact or electrode array 146 for delivery of electrical stimulation (current) to the recipient's cochlea 145.

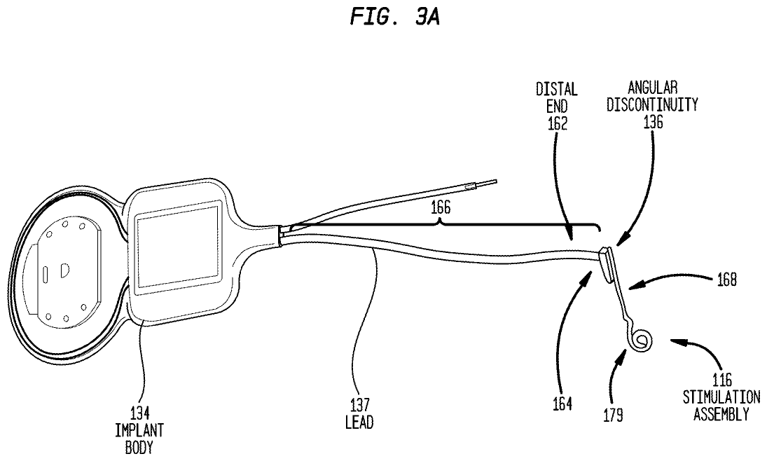

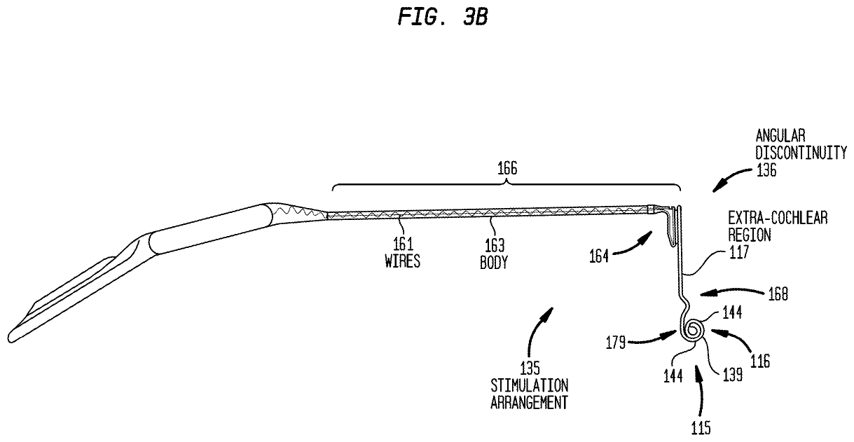

Stimulation assembly 116 extends through an opening 147 in the recipient's cochlea 145 (e.g., cochleostomy, the round window, etc.) and has a proximal end 168 connected to the angular discontinuity 136, which in turn is connected to the lead 137. A plurality of conductors/wires 161 (shown in FIG. 3B) extend through a resiliently flexible body 163 (FIG. 3B) formed, for example, from silicone, of the lead 137 and through a hermetic feedthrough (not shown in FIG. 1D) to electrically couple the electrodes 144 to the stimulator unit 142. The implantable component 112 also includes an electrode outside of the cochlea 145, sometimes referred to as the extra-cochlear electrode (ECE) 139.

As noted, the cochlear implant system 102 includes the external coil 108 and the implantable coil 114. The external magnet 152 is fixed relative to the external coil 108 and the implantable magnet 152 is fixed relative to the implantable coil 114. The magnets fixed relative to the external coil 108 and the implantable coil 114 facilitate the operational alignment of the external coil 108 with the implantable coil 114. This operational alignment of the coils enables the external component 104 to transmit data and power to the implantable component 112 via a closely-coupled wireless RF link 131 formed between the external coil 108 with the implantable coil 114. In certain examples, the closely-coupled wireless link 131 is a radio frequency (RF) link. However, various other types of energy transfer, such as infrared (IR), electromagnetic, capacitive and inductive transfer, may be used to transfer the power and/or data from an external component to an implantable component and, as such, FIG. 1D illustrates only one example arrangement.

As noted above, sound processing unit 106 includes the external sound processing module 124. The external sound processing module 124 is configured to convert received input signals (received at one or more of the input devices 113) into output signals for use in stimulating a first ear of a recipient (i.e., the external sound processing module 124 is configured to perform sound processing on input signals received at the sound processing unit 106). Stated differently, the one or more processors in the external sound processing module 124 are configured to execute sound processing logic in memory to convert the received input signals into output signals that represent electrical stimulation for delivery to the recipient.

As noted, FIG. 1D illustrates an embodiment in which the external sound processing module 124 in the sound processing unit 106 generates the output signals. In an alternative embodiment, the sound processing unit 106 can send less processed information (e.g., audio data) to the implantable component 112 and the sound processing operations (e.g., conversion of sounds to output signals) can be performed by a processor within the implantable component 112.

Returning to the specific example of FIG. 1D, the output signals are provided to the RF transceiver 122, which transcutaneously transfers the output signals (e.g., in an encoded manner) to the implantable component 112 via external coil 108 and implantable coil 114. That is, the output signals are received at the RF interface circuitry 140 via implantable coil 114 and provided to the stimulator unit 142. The stimulator unit 142 is configured to utilize the output signals to generate electrical stimulation signals (e.g., current signals) for delivery to the recipient's cochlea. In this way, cochlear implant system 102 electrically stimulates the recipient's auditory nerve cells, bypassing absent or defective hair cells that normally transduce acoustic vibrations into neural activity, in a manner that causes the recipient to perceive one or more components of the received sound signals.

As detailed above, in the external hearing mode the cochlear implant 112 receives processed sound signals from the sound processing unit 106. However, in the invisible hearing mode, the cochlear implant 112 is configured to capture and process sound signals for use in electrically stimulating the recipient's auditory nerve cells. In particular, as shown in FIG. 1D, the cochlear implant 112 includes a plurality of implantable sensors 153 and an implantable sound processing module 158. Similar to the external sound processing module 124, the implantable sound processing module 158 may comprise, for example, one or more processors and a memory device (memory) that includes sound processing logic. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

In the invisible hearing mode, the implantable sensors 153 are configured to detect/capture signals (e.g., acoustic sound signals, vibrations, etc.), which are provided to the implantable sound processing module 158. The implantable sound processing module 158 is configured to convert received input signals (received at one or more of the implantable sensors 153) into output signals for use in stimulating the first ear of a recipient (i.e., the processing module 158 is configured to perform sound processing operations). Stated differently, the one or more processors in implantable sound processing module 158 are configured to execute sound processing logic in memory to convert the received input signals into output signals 155 that are provided to the stimulator unit 142. The stimulator unit 142 is configured to utilize the output signals 155 to generate electrical stimulation signals (e.g., current signals) for delivery to the recipient's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 could operate differently in different embodiments. For example, in one alternative implementation of the external hearing mode, the cochlear implant 112 could use signals captured by the sound input devices 118 and the implantable sensors 153 in generating stimulation signals for delivery to the recipient.

As noted above, the cochlear implant 112 comprises implantable sensors 153. In certain embodiments, the implantable sensors 153 comprise at least two sensors 156 and 160, where at least one of the sensors is designed to be more sensitive to bone-transmitted vibrations then to acoustic (air-borne) sound waves. In the illustrative embodiment of FIG. 1D, the implantable sensor 156 is an implantable “sound” sensor/transducer that is primarily configured to detect/receive external acoustic sounds (e.g., an implantable microphone), while the implantable sensor 160 is a “vibration” sensor that is primarily configured to detect/receive internal vibration signals, including body noises. (e.g., another implantable microphone or an accelerometer). These sensors can take a variety of different forms, such as another implantable microphone, an accelerometer, etc. However, for ease of description, embodiments presented herein will be primarily described with reference to the use of an implantable microphone as the sound sensor and an accelerometer as the vibration sensor. The increased sensitivity of the accelerometer to vibration signals (e.g., body noises) may be due to, for example, the structure of the accelerometer relative to the microphone, the implanted position of the accelerometer relative to the microphone, etc. For example, in certain embodiments, the accelerometer and the microphone are structurally similar, but they are placed in different locations which accounts for the vibration/body noise sensitivity difference. Again, it is to be appreciated that these specific implementations are non-limiting and that embodiments of the present invention may be used with different types of implantable sensors

The implantable microphone 156 and the accelerometer 160 can each be disposed in, or electrically connected to, the implant body 134. In operation, the implantable microphone 156 and the accelerometer 160 each detect input signals and convert the detected input signals into electrical signals. The input signals detected by the implantable microphone 156 and the accelerometer 160 can each include external acoustic sounds and/or vibration signals, including body noises.

As noted, the stimulation assembly 116 is a configured to be at least partially implanted into the cochlea 145 and the implant body 134 is configured to be implanted some distance away from the cochlea 145, typically at the outer surface of the recipient's skull. Also as noted, the electrodes 144 of the stimulation assembly 116 are electrically connected to the stimulator unit 142 in the implant body 134 via wires 161 (FIG. 3B) extending through the lead 137.

The implanted position of the implant body 134, which includes the stimulator unit 142, can vary with different surgical approaches and different head sizes of different, meaning that the final distance between an the implant body 134 and the stimulation assembly 116 can vary. Accordingly, the lead 137 is intentionally manufactured so as to be sufficiently long to accommodate different final distances between the implant body 134 and the stimulation assembly 116. During surgery, the excess length of the lead 137 can be “coiled” or otherwise physically manipulated for positioning within the head of the recipient. As noted above, lead 137 of FIG. 1D terminates in an angular discontinuity 136 that is configured to substantially isolate the stimulation assembly 116 from the lead 137. That is, the angular discontinuity 136 minimizes movement and/or twisting of the stimulation assembly 116 during physical manipulation of the lead 137 (e.g., the angular discontinuity 136 mechanically decouples the lead 137 from the intra-cochlear stimulation assembly 116 to minimize transmission of forces and torque from the lead to the stimulation assembly). As such, the manipulation (e.g., coiling) of the excess length of the lead 137 does not cause corresponding movement and/or twisting of the stimulation assembly 116 from its intended position which, in turn, minimizes damage to intra-cochlear structures and/or displacement or over insertion of the stimulation assembly 116.

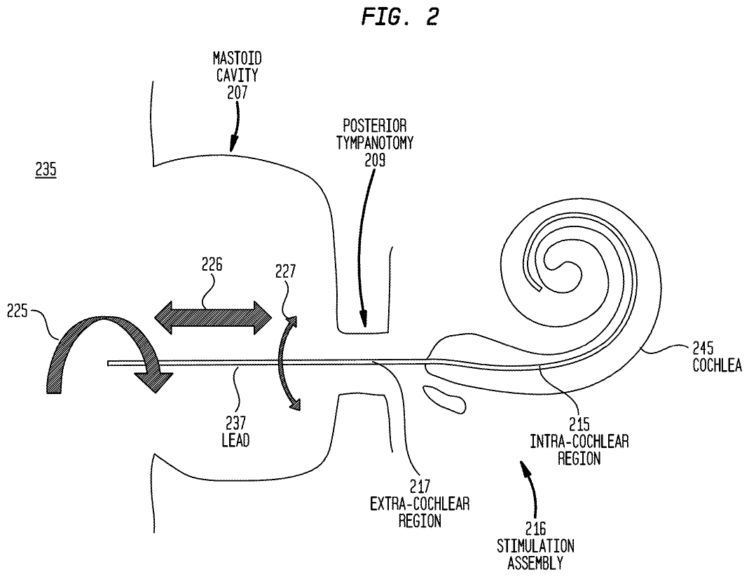

FIG. 2 illustrates a conventional stimulation arrangement 235 comprising a lead 237 and a stimulation assembly 216 having an intra-cochlear region 215 and an extra-cochlear region 217. The lead 237 is configured to be positioned in a surgically formed mastoid cavity 207, while the stimulation assembly 216 (e.g., intra-cochlear region 215) is configured to be implanted in the cochlea 245. The extra-cochlear region 217 extends from the lead 237 to the intra-cochlear region 215 through the posterior tympanotomy 209. In this example, the lead 237 is mechanically coupled to the extra-cochlear region 217. As such, torsional forces 225, linear forces 226, and/or angular forces 227 applied to the lead 237 are transferred to the stimulation assembly 216.

In contrast to the arrangement of FIG. 2, FIGS. 3A and 3B are diagrams illustrating further details of the decoupling structure of FIGS. 1A-1D in the form of the angular discontinuity 136 of stimulation arrangement 135, in accordance with certain embodiments presented. More specifically, FIG. 3A is a perspective view of the cochlear implant system 102, while FIG. 3B is a side-view of the cochlear implant system 102. For ease of reference, FIGS. 3A and 3B will be described together and with reference to stimulation arrangement 135 of the cochlear implant system 102.

In the example of FIGS. 3A and 3B, the angular discontinuity 136 is formed between a distal end 162 of the lead 137 and a proximal end 168 of the stimulation assembly 116. In this example, the extra-cochlear region 117 has a length such that the angular discontinuity 136 is located with the mastoid cavity. However, it is to be appreciated that the angular discontinuity 136 could alternatively be located closer to the cochlea 145.

The angular discontinuity 136 comprises a pre-formed/pre-biased bend 164 (e.g., molded bend), where the pre-formed bend 164 is coupled with a proximal extension 166 extending a minimum proximal distance from the pre-formed bend 164. In the example of FIGS. 3A-3C, the pre-formed bend 164 is a substantially 90 degree bend and, as a result, the proximal extension 166 is oriented substantially orthogonal to an axis 177 of a straight section 179 of the intra-cochlear region 115 within the basal turn of the cochlea 145. The pre-formed bend 164 can be to the inferior side or the superior side of the lead 137. As used herein, “pre-formed” generally means a shape a material will retain, or naturally return to, in an unstressed state (e.g., in the absence of externally applied forces).

The extension 166 is referred to as a “proximal” extension because the extension is located on an opposite side of the pre-formed bend 164 from the stimulation assembly 116. Moreover, the proximal extension 166 has a minimum length. The minimum length of the proximal extension 164, when combined with the pre-formed bend 164, functions to mechanically decouple the lead 137 from the extra-cochlear region 117 and the stimulation assembly 116. That is, the angular discontinuity 136 minimizes torsional, linear, and/or angular forces applied to the lead 137 from being transferred to the stimulation assembly 116.

A stimulation arrangement, such as stimulation arrangement 135, is a complex system in which the lead 137 is, at times, simultaneously exposed to linear, angular and torsional forces. A straight lead (without the angular discontinuity 136) tends to push the stimulation inwards and acts as a lever on the intra-cochlear region. In addition, it converts torsional force directly to the intra-cochlear region even when the lead is coiled and partly because the lead is coiled. The angular discontinuity 136 works in a variety of ways to prevent the various forces from being transmitted directly to the intra-cochlear section. It also provides the surgeon with a clear indication whether the stimulation assembly 116 is being forced, and gives the surgeon an easier system to ensure that manipulation and coiling do not convey forces to the inner ear.

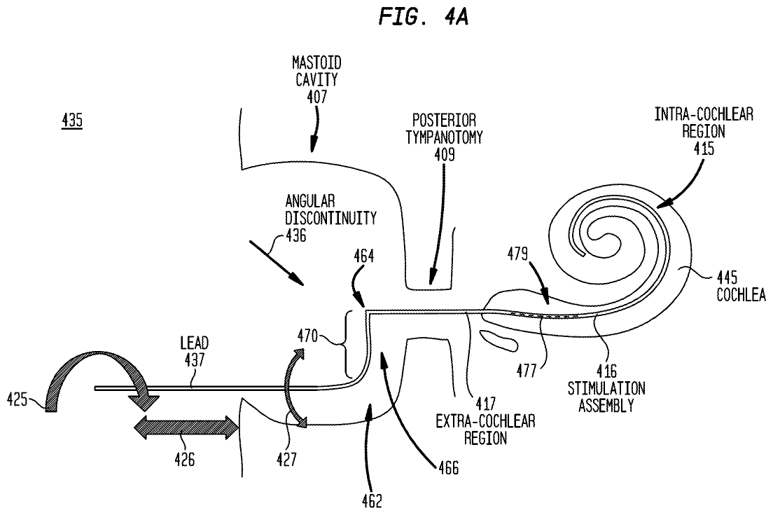

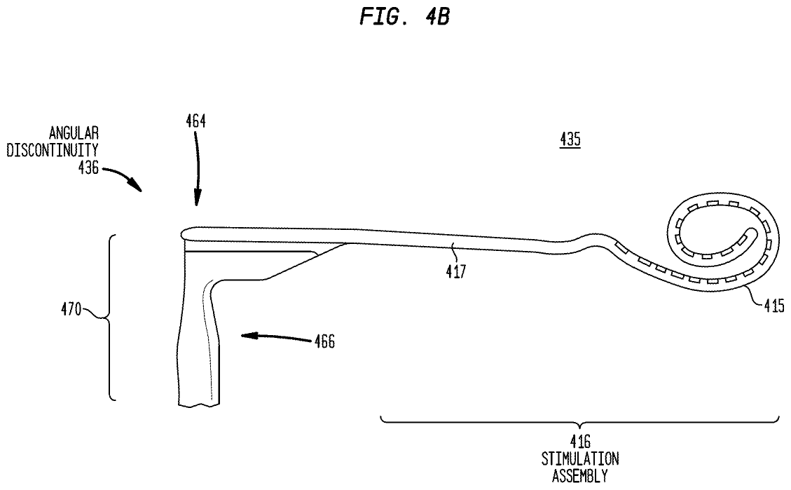

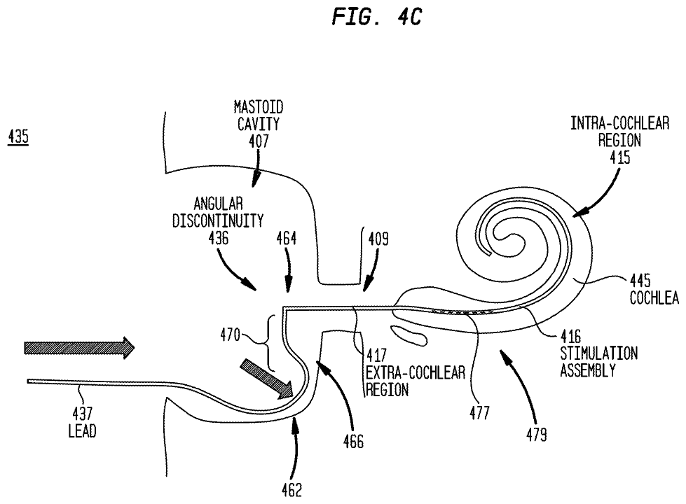

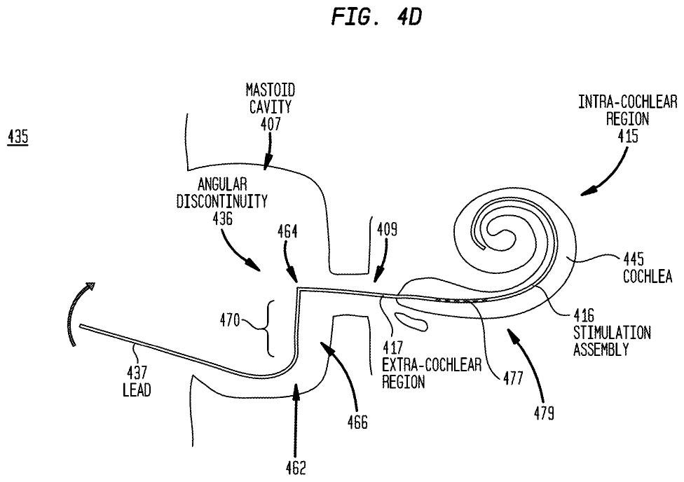

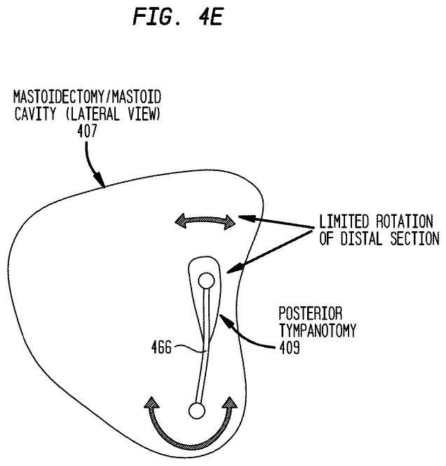

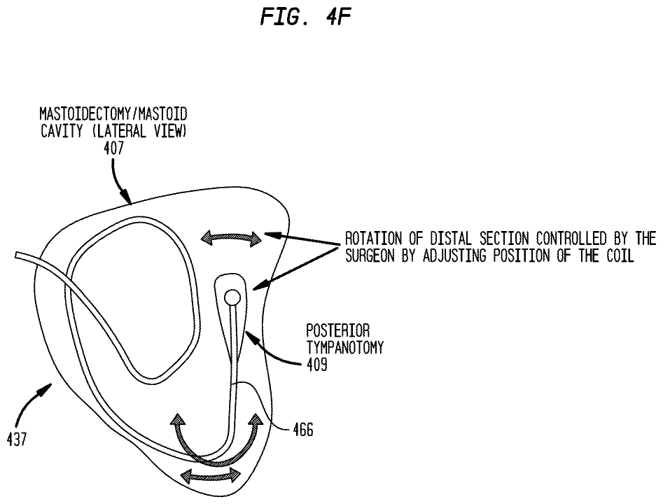

As noted, the pre-formed bend 164 is a substantially 90 degree bend (e.g., the proximal extension 166 is oriented substantially orthogonal to an axis 177 of a straight section 179 of the intra-cochlear region 115 within the basal turn of the cochlea 145). As described further below with reference to FIGS. 4A-4F, the 90 degree orientation of the proximal extension 166 means that any residual torque in the lead 137 is applied in a direction that is more readily reacted by the stimulation arrangement 135 within the facial recess, and does not result in twisting of the stimulation assembly 116. For example, the forces and torques applied to the lead 137 are reacted against the floor and walls of the mastoidectomy and against the walls of the facial recess rather than being transmitted directly to the stimulation assembly 116. This is a key function of the proximal extension

To be effective in minimizing transmission of force and torque from the lead 137 to the stimulation assembly 116, the proximal extension 166 (i.e., the region that is at approximately 90 degrees to axis 177 of the straight section 179 of the intra-cochlear region 115 within the basal turn of the cochlea 145) has a certain minimum length. In the example of FIGS. 3A and 3B, the length of the proximal extension 166 is equal to the length of the lead 237 so that the entire lead is oriented at 90 degrees relative to the electrode axis in its undeformed/unstressed state (e.g., no applied external forces), in which case the surgeon bends the lead to suit the anatomy of the patient

As noted, FIGS. 3A and 3B show that the proximal extension 166 extends the entirety of the lead 137. That is, the entire lead 137 is oriented substantially 90 degrees to the axis 177 of the straight section 179 of the intra-cochlear region 115 within the basal turn of the cochlea 145. In this example, the lead 137 can act as a stabilizing feature when arranged so that it contacts the walls of the mastoid cavity near the mastoid tip and stability is further improved if the lead is malleable in nature rather than elastic. The 90 degree lead orientation provides a clear visual indication to the surgeon of the plane of symmetry of the stimulation arrangement 135 relative to the anatomy and the 90 degree lead orientation naturally takes the lead 137 away from the line of vision of the surgeon during the insertion. In addition, the 90 degree lead orientation enhances the visibility of any markings placed on the proximal side of the electrode handle which indicate the electrode insertion depth. As described further below, the 90 degree bend 164 prevents transmission of torsion to the intra-cochlear region, and as well as provides a good indication of any rotation of the intra-cochlear region, it is easier for the surgeon to manage this twisting when coiling the lead by allowing the surgeon to adjust the radius or position of the coiled lead, so the 90 degree component is kept pointing in the correct direction.

An arrangement in which the entire lead is oriented at 90 degrees, as shown in FIGS. 3A and 3B, is one advantageous implementation of an angular discontinuity. However, in certain embodiments, the entire lead is not necessarily orientated at 90 degrees and, instead, the proximal extension extends a minimum distance after which the lead can assume a different orientation. FIGS. 4A-4F illustrate one such example.

More specifically, FIG. 4A is schematic diagram illustrating a stimulation arrangement 435 comprising an angular discontinuity 436, while FIG. 4B is a side-view of only a portion of the stimulation arrangement 435. For ease of description, FIGS. 4A and 4B will be described together. FIGS. 4C, 4D, 4E, and 4F are schematic diagram illustrating one example of the stimulation arrangement 435 in which the angular discontinuity 436 operates to minimize transfer of forces from a lead to a stimulation assembly, in accordance with certain embodiments presented herein.

Referred first to FIGS. 4A and 4B, the stimulation arrangement 435 comprises a lead 437 and a stimulation assembly 416 having an intra-cochlear region 415 and an extra-cochlear region 417. The lead 437 is configured to be positioned in a surgically formed mastoid cavity 407, while the stimulation assembly 416 is configured to be implanted in the cochlea 445 (e.g., intra-cochlear region 415). The extra-cochlear region 417 extends through the posterior tympanotomy 409.

In the example of FIGS. 4A and 4B, the angular discontinuity 436 is formed between the lead and the extra-cochlear region 417. The angular discontinuity 436 comprises a substantially 90 degree pre-formed/pre-biased (e.g., molded) bend 464, where the pre-formed bend 464 is coupled with a proximal extension 466 extending a minimum distance 470 from the pre-formed bend 464 (i.e., the proximal extension 466 has a minimum length 470). The proximal extension 466 comprises a section of material that is pre-biased (e.g., pre-molded) so as to be, in an unstressed state (e.g., no applied external forces), oriented substantially 90 degrees relative to an axis 477 of a straight section 479 of the intra-cochlear region 415 within the basal turn of the cochlea 445

As noted, in the example of FIGS. 4A and 4B, the proximal extension 466 extends a distance 470 from the pre-formed bend 464. That is, the proximal extension 466 has a length 470 that is oriented substantially 90 degrees relative to an axis 477 of a straight section 479 of the intra-cochlear region 415 within the basal turn of the cochlea 445 (e.g., the proximal extension 466 remains at approximately 90 degrees in an unstressed (undeformed) state).

The 90 degree orientation and length 470 of the proximal extension 466 means that any residual torque in the lead 437 is applied in a direction that is more readily reacted by the stimulation arrangement 435 within the facial recess, and does not result in twisting of the stimulation assembly 416 (e.g., the angular discontinuity 426 minimizes the transfer of torsional forces 435, linear forces 426, and/or angular forces 427 to the stimulation assembly 416). This is shown in greater detail in FIGS. 4C-4F.

To be effective in minimizing transmission of force and torque from the lead 437 to the stimulation assembly 416, the proximal extension 466 (i.e., the region that is at approximately 90 degrees) has a certain minimum length. At a minimum, the length 470 of the proximal extension 466 is about at least five times the maximum diameter of the stimulation assembly 416, and preferably more than ten times the maximum diameter of the stimulation assembly 416. For example, assuming the stimulation assembly 416 has a diameter of 0.5 mm, the proximal extension 466 has a length of at least approximately 2.5 mm or, in certain embodiments, a length of at least approximately 5 mm.

In certain embodiments, the pre-formed bend 464 is configured to retain the predetermined angle during and after implantation of the stimulation assembly 416. However, in certain embodiments, the pre-formed bend 464 can be at least partially straightened during and after implantation of the stimulation assembly 416 (e.g., via use of a stiffening element, such as a sheath). In such embodiments, the pre-formed bend 464 is substantially resilient so as to return to the predetermined angle after insertion (e.g., after the predetermined angle is removed or deactivated).

As noted, FIGS. 4C-4F illustrate further details of how the 90 degree orientation and length 470 of the proximal extension 466 means that any residual torque in the lead 437 is applied in a direction that is more readily reacted by the stimulation arrangement 435 within the facial recess, and does not result in twisting of the stimulation assembly 416. That is, the pre-formed bend 464 and the proximal extension 466 create an offset between the stimulation assembly 416 and lead 437 (the part handled by the surgeon), which allows forces imparted to the lead 437 to reacted against the walls of the mastoidectomy and the walls of the facial recess to produce a ‘de-couple’ that reacts torque, and limits movement.

More specifically, FIG. 4C illustrates an embodiment in which the lead 437 is pushed inward and, as a result of the angular discontinuity, the lead 437 bends (e.g., at or near the proximal extension 466) and, as such, the lead is unable to convey force sufficient to overcome the stabilization mechanism (e.g., packing) 475 at the opening to the inner ear/cochlea 445. In this example, the floor of the mastoidectomy (mastoid cavity 407) provides a base which also limits extension of the lead 437 in the direction of the inner ear.

FIG. 4D illustrates an example in which the lead 437 is superiorly, which due to the angular discontinuity 436, results in a bend in the lead in rather than conveying the force directly to the stimulation assembly 416 416 in the cochlea 445. FIG. 4E illustrates a lateral view of the mastoid cavity 407 and the proximal extension 466. In this example, twisting of the lead 437 lead cannot twist the stimulation assembly 416 since it is contained by the right angle shape of the angular discontinuity 436 and the posterior tympanotomy.

FIG. 4F illustrates a lateral view of the mastoid cavity 407 and the proximal extension 466. In this example, when coiling the lead 437, the surgeon can see whether the shape of the coil is causing the intra-cochlear region 415 to torsion and rotate. The surgeon can then easily adjust the position of the coiled lead 437 to avoid torsioning and rotation of the intra-cochlear region 415. After coiling, the surgeon can avoid torsion on the intra-cochlear region 415 by adjusting the position of the coiled lead 437.

In general, FIGS. 4C-4F illustrate show how the isolating structure prevents transmission of torsion and movement of the proximal end of the lead from transmitting force and torsion to the intra-cochlear region. The stimulation arrangement 435, and other stimulation arrangements, is a system in which the lead is simultaneously exposed to linear, angular and torsional forces. A straight lead (without discontinuity) tends to push the stimulation assembly 416 inward and acts as a lever on the internal section. In addition, it converts torsional force directly to the intra-cochlear region 415 even when the lead 437 is coiled and partly because the lead is coiled. The angular discontinuity works in a variety of ways to prevent the various forces from being transmitted directly to the intracochlear section. It also provides the surgeon with a clear indication whether the electrode is being forced, and gives the surgeon an easier system to ensure that manipulation and coiling do not convey forces to the inner ear.

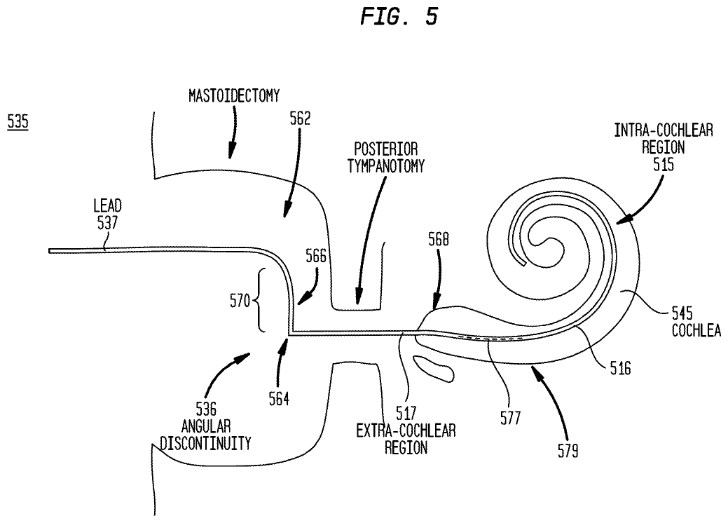

FIGS. 4A-4F illustrate an example in which the pre-formed bend 464 of the angular discontinuity 436 is to the inferior side of the stimulation arrangement 435. In accordance with embodiments presented herein, a pre-formed bend could alternatively be to the superior side of the stimulation arrangement, such as shown in FIG. 5. That is, FIG. 5 illustrates a stimulation arrangement 535 comprising a lead 537 and a stimulation assembly 516 having an intra-cochlear region 515 and an extra-cochlear region 517. An angular discontinuity 536 is formed between the distal end 562 of the lead 537 and the proximal end 568 of the stimulation assembly 516, where the angular discontinuity 536 comprises a substantially 90 degree pre-formed/pre-biased (e.g., molded) bend 564, where the pre-formed bend 564 is coupled with a proximal extension 566 extending a minimum distance 570 from the pre-formed bend 564. In this example, the proximal extension 566 comprises a section of material that is pre-formed (e.g., molded) so as to be, in an unstressed state, oriented substantially 90 degrees relative to an axis 577 of a straight section 579 of the intra-cochlear region 515 within the basal turn of the cochlea 545. In contrast to FIGS. 4A and 4B, the pre-formed bend 564 is to the superior side, rather than the inferior side. In other embodiments, the pre-formed bend could be oriented to in other (e.g., lateral) directions.

It is also noted that is a lead exits an angular discontinuity in the plane of symmetry of the stimulation assembly, then it is equally compatible with left or right ears (e.g., by selecting an angle to be either inferior or superior direction the one device can be used in left and right ears as noted in the text below). The lead may exit to the side that corresponds to the superior side or to the inferior side (relative to the recipient) when the electrode is inserted in the cochlea. The pre-formed bend could alternatively be to the posterior direction. Another possibility is to create a malleable lead that allows the surgeon to bend the lead to create the shape that they prefer. This could be facilitated by a bending tool.

In certain embodiments, such as those shown in FIGS. 3A-3B, 4A-4B, and 5, the proximal extension can be generally straight (e.g., a generally linear shape/arrangement). In alternative embodiments, the proximal extension can comprise a non-linear arrangement (e.g., comprise one or more bends). For example, in certain embodiments, the proximal extension can comprise a single-turn or multiple-turn coil, a serpentine shape, a zig-zag shape, an undulating shape (e.g., a flat ribbon shape, a flat ribbon shape with a twist to, for example, allow flexing in multiple angles, etc.), include a malleable shape element, combinations thereof, etc.

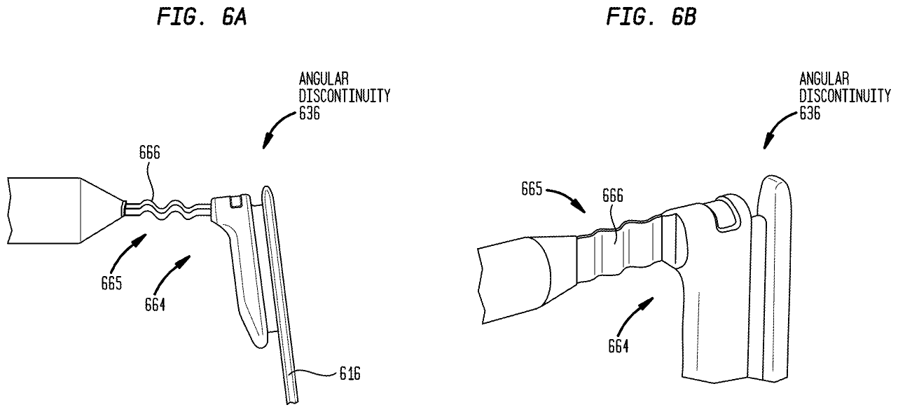

FIGS. 6A and 6B are side and perspective views, respectively, of an angular discontinuity 636 comprising a non-linear proximal extension, in accordance with certain embodiments presented herein. More specifically, angular discontinuity 636 includes a substantially 90 degree pre-formed/pre-biased bend 664 and a proximal extension 666 extending a minimum distance from the pre-formed bend 664. In this example, the proximal extension 666 has a pre-molded undulating shape 665 (e.g., vertical or horizontal waves to remove lead torsion force from stimulation assembly 616) that, in an unstressed state, is oriented substantially 90 degrees relative to an axis of straight portion of a distal section of the stimulation assembly 616 within the cochlea of the recipient.

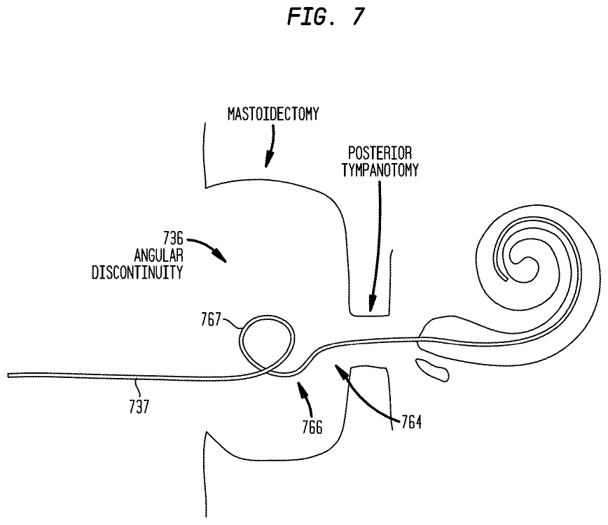

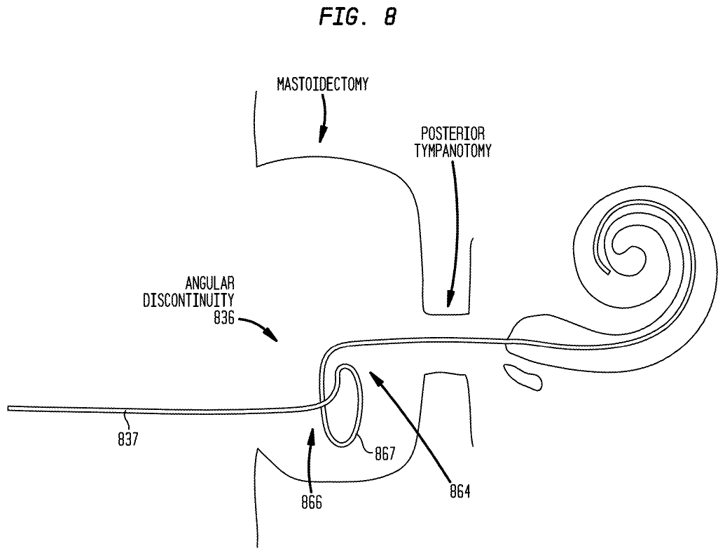

FIG. 7 is a schematic diagram illustrating an angular discontinuity 736 comprising another non-linear proximal extension, in accordance with certain embodiments presented herein. As shown, the angular discontinuity 736 comprises a pre-formed/pre-biased bend 764 in lead 737 and a proximal extension 766 extending a minimum distance from the pre-formed bend 764. In this example, the proximal extension 766 includes at least one pre-molded loop 767 having an orientation generally in the plane of with the lead 737 (e.g., one or more loops perpendicular to the floor of mastoidectomy).

FIG. 8 is a schematic diagram illustrating another angular discontinuity 836 comprising another non-linear proximal extension, in accordance with certain embodiments presented herein. As shown, the angular discontinuity 836 comprises a pre-formed/pre-biased bend 864 in lead 837 and a proximal extension 866 extending a minimum distance from the pre-formed bend 864. In this example, the proximal extension 866 includes at least one pre-molded loop 867 having an orientation generally perpendicular/orthogonal to the plane of the lead 837 (e.g., one or more loops parallel to the floor of the mastoidectomy).

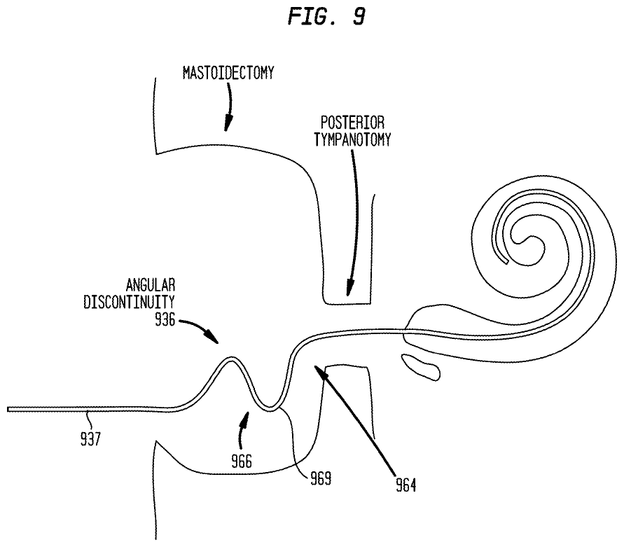

FIG. 9 is a schematic diagram illustrating another angular discontinuity 936 comprising another non-linear proximal extension, in accordance with certain embodiments presented herein. As shown, the angular discontinuity 936 comprises a pre-formed/pre-biased bend 964 in lead 937 and a proximal extension 966 extending a minimum distance from the pre-formed bend 964. In this example, the proximal extension 966 includes a serpentine structure 969 in the plane of the lead 937.

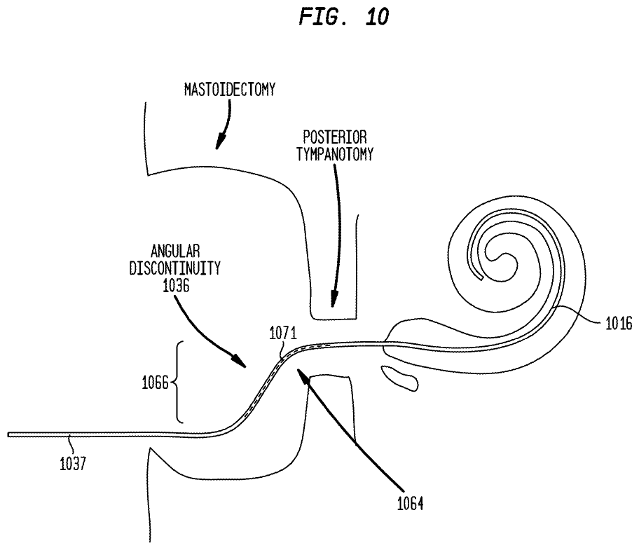

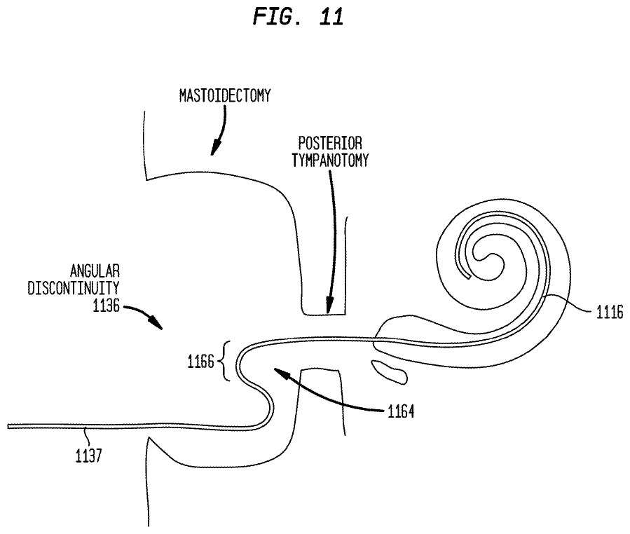

Various embodiments described above illustrate angular discontinuities comprising substantially 90 degree pre-formed bends. The 90 degree bend may be beneficial, but it is to be appreciated that certain embodiments may include pre-formed bends within a range of angle greater or less than 90 degrees. For example, angular discontinuities in accordance with certain embodiments presented herein can, in include pre-formed bends in the range of approximately 45 degrees to approximately 120 degrees relative to an axis of a straight section of the intra-cochlear region within the basal turn of the cochlea. FIG. 10 illustrates an example including a pre-formed bend that is greater than 90 degrees, while FIG. 11 illustrates an example including a pre-formed bend that is less than 90 degrees.

More specifically, FIG. 10 is a schematic diagram illustrating another angular discontinuity 1036 comprising another non-linear proximal extension, in accordance with certain embodiments presented herein. As shown, the angular discontinuity 1036 comprises a pre-formed/pre-biased bend 1064 in lead 1037 and a proximal extension 1066 extending a minimum distance from the pre-formed bend 1064. In this example, pre-formed bend 1064 has an angle that is greater than 90 degrees, relative to an axis of a straight section of the elongate stimulation assembly 1016 within a basal turn of the cochlea.

FIG. 10 also illustrates the use of a pre-biased shape element 1071 disposed in a resiliently flexible material 1073 forming the angular discontinuity 1036. In this example, the pre-biased shape element 1071 is pre-formed to have the predetermined angle and to form the length of the proximal extension 1066. In certain such embodiments, the pre-biased shape element 1071 is configured to be straightened during implantation of the elongate stimulation assembly 1016, and is configured to adopt the predetermined angle following implantation of the elongate stimulation assembly.

It is to be appreciated that the pre-biased shape element 1071 shown in FIG. 10 is merely illustrative. In other embodiments, a pre-biased shape element could be used to form only the pre-formed bend or only the proximal extension.

FIG. 11 is a schematic diagram illustrating another angular discontinuity 1136 comprising another non-linear proximal extension, in accordance with certain embodiments presented herein. As shown, the angular discontinuity 1136 comprises a pre-formed/pre-biased bend 1164 in lead 1137 and a proximal extension 1166 extending a minimum distance from the pre-formed bend 1164. In this example, pre-formed bend 1164 has an angle that is less than 90 degrees, relative to an axis of a straight section of the elongate stimulation assembly 1116 within a basal turn of the cochlea.

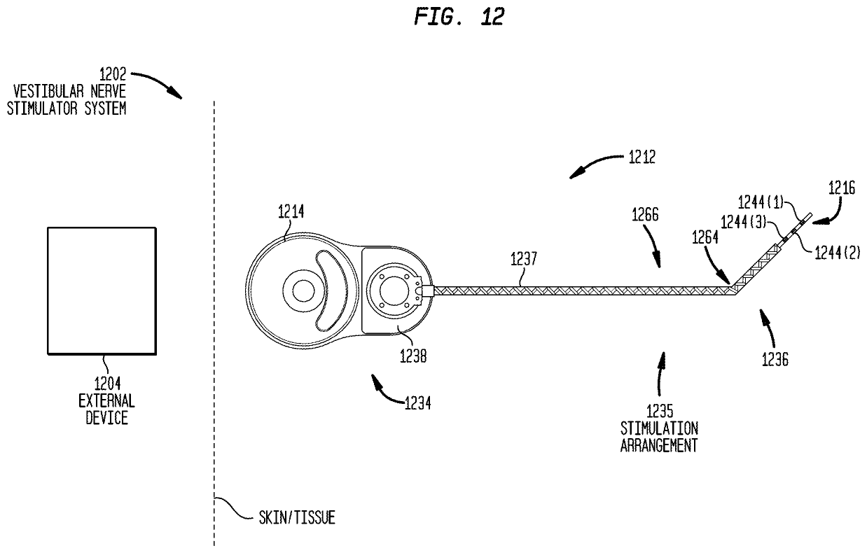

Decoupling structures in accordance with embodiments presented herein have generally been described with reference to cochlear stimulation arrangements. However, as noted above, the decoupling structures can be used with a number of different stimulation arrangements for implantation at other locations within a recipient. For example, FIG. 12 illustrates an example vestibular stimulator system 1202, with which embodiments presented herein can be implemented. As shown, the vestibular stimulator system 1202 comprises an implantable component (vestibular stimulator) 1212 and an external device/component 1204 (e.g., external processing device, battery charger, remote control, etc.). The external device 1204 is configured to, for example, transfer power and/or data to the vestibular stimulator 1212.

The vestibular stimulator 1212 comprises an implant body (main module) 1234 and a stimulation arrangement 1235 comprising a lead 1237 and a stimulating assembly 1216, all configured to be implanted under the skin/tissue (tissue) of the recipient. The implant body 1234 generally comprises a hermetically-sealed housing 1238 in which RF interface circuitry, one or more rechargeable batteries, one or more processors, and a stimulator unit are disposed. The implant body 1234 also includes an internal/implantable coil 1014 that is generally external to the housing 1238, but which is connected to the transceiver via a hermetic feedthrough (not shown).

The stimulating assembly 1216 comprises a plurality of electrodes 1024(1)-(3) disposed in a carrier member (e.g., a flexible silicone body). In this specific example, the stimulating assembly 1216 comprises three (3) stimulation electrodes, referred to as stimulation electrodes 1244(1), 1244(2), and 1244(3). The stimulation electrodes 1244(1), 1244(2), and 1244(3) function as an electrical interface for delivery of electrical stimulation signals to the recipient's vestibular system.

The stimulating assembly 1216 is configured such that a surgeon can implant the stimulating assembly adjacent the recipient's otolith organs via, for example, the recipient's oval window. It is to be appreciated that this specific embodiment with three stimulation electrodes is merely illustrative and that the techniques presented herein may be used with stimulating assemblies having different numbers of stimulation electrodes, stimulating assemblies having different lengths, etc.

As shown, a decoupling structure in the form of an angular discontinuity 1236 connects a distal end of the lead 1237 to a proximal end of the stimulation assembly 1216. The angular discontinuity 1236 comprises a section of material that is pre-formed/pre-biased (e.g., molded) into a bend 1264, where the pre-formed bend 1264 is coupled with a proximal extension 1266 extending a minimum proximal distance from the pre-formed bend 1264. In the example of FIG. 12, the pre-formed bend 1264 is a bend of greater than 90 degrees.

The extension 1266 is referred to as a “proximal” extension because the extension is located on an opposite side of the pre-formed bend 1264 from the stimulation assembly 1216. Moreover, the proximal extension 1266 has a minimum length. The minimum length of the proximal extension 1264, when combined with the pre-formed bend 1264, functions to mechanically decouple the lead 1327 from the stimulation assembly 1216. That is, the angular discontinuity 1236 minimizes torsional, linear, and/or angular forces applied to the lead 1237 from being transferred to the stimulation assembly 1216. In the example of FIG. 12, the length of the proximal extension 1266 is equal to the length of the lead 1237 (e.g., the proximal extension 1266 extends the entirely of the lead 1237).

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments may be combined with another in any of a number of different manners.

1. An implantable medical device, comprising:
a stimulator unit configured to be implanted in a recipient;
an elongate stimulation assembly configured to be implanted in the recipient, wherein the elongate stimulation assembly includes at least one electrode;
a lead comprising a resiliently flexible body and at least one wire disposed in the resiliently flexible body and electrically connecting the at least one electrode to the stimulator unit; and
a decoupling structure physically connecting a proximal end of the elongate stimulation assembly to a distal end of the lead, wherein the decoupling structure mechanically decouples the elongate stimulation assembly from the lead.

2. The implantable medical device of claim 1, wherein the decoupling structure is configured to minimize transfer of torsional, linear, and angular forces applied to the lead from being transferred to the elongate stimulation assembly.

3. The implantable medical device of claim 1, wherein the decoupling structure comprises a pre-formed bend and a proximal extension having a predetermined first length oriented at a predetermined angle relative to an axis of a straight portion of a distal section of the elongate stimulation assembly within a body chamber of the recipient.

4. The implantable medical device of claim 3, wherein the pre-formed bend comprises an angle in a range of approximately 45 degrees to approximately 120 degrees relative to an axis of a straight portion of a distal section of the elongate stimulation assembly within a body chamber of the recipient.

5. The implantable medical device of claim 4, wherein the elongate stimulation assembly is configured to be implanted in a cochlea of the recipient, and wherein the angle in the range of approximately 45 degrees to approximately 120 degrees relative to an axis of a straight section of the elongate stimulation assembly within a basal turn of the cochlea.

6. The implantable medical device of claim 3, wherein the pre-formed bend comprises an angle of approximately 90 degrees relative to an axis of a straight portion of a distal section of the elongate stimulation assembly within a body chamber of the recipient.

7. The implantable medical device of claim 3, wherein the pre-formed bend comprises an angle of between approximately 90 degrees and approximately 120 degrees relative to an axis of a straight portion of a distal section of the elongate stimulation assembly within a body chamber of the recipient.

8. The implantable medical device of claim 3, wherein the pre-formed bend comprises an angle of between approximately 45 degrees and approximately 90 degrees relative to an axis of a straight portion of a distal section of the elongate stimulation assembly within a body chamber of the recipient.

9. The implantable medical device of claim 3, wherein the pre-formed bend is formed by a moldable section of material.

10. The implantable medical device of claim 3, wherein the pre-formed bend is formed by a section of material that is pre-molded into the predetermined angle.

11. (canceled)

12. The implantable medical device of claim 3, wherein the pre-formed bend is formed by a pre-biased shape element disposed in a section of resiliently flexible material.

13. The implantable medical device of claim 12, wherein the pre-biased shape element is configured to be at least partially straightened during implantation of the elongate stimulation assembly into the recipient, and is configured to adopt the predetermined angle following implantation of the elongate stimulation assembly into the recipient.

14. The implantable medical device of claim 3, wherein, in an unstressed state, substantially an entire length of the lead is oriented at the predetermined angle relative to an axis of a straight portion of a distal section of the elongate stimulation assembly within a body chamber.

15. The implantable medical device of claim 3, wherein the proximal end of the elongate stimulation assembly has a diameter, and wherein the predetermined first length is at least five times the diameter of the elongate stimulation assembly.

16. The implantable medical device of claim 15, wherein the predetermined first length is at least ten times the diameter of the elongate stimulation assembly.

17. The implantable medical device of claim 3, wherein the predetermined first length is at least 2.5 mm.

18. The implantable medical device of claim 3, wherein the predetermined first length is at least 5 mm.

19. The implantable medical device of claim 3, wherein the proximal extension has a linear shape extending the predetermined first length.

20. The implantable medical device of claim 3, wherein the proximal extension has a pre-formed non-linear shape within the predetermined first length.

21. The implantable medical device of claim 20, wherein the pre-formed non-linear shape comprises at least one pre-formed coil.

22. The implantable medical device of claim 20, wherein the pre-formed non-linear shape comprises an undulating shape.

23. The implantable medical device of claim 20, wherein the pre-formed non-linear shape comprises at least one of a zig-zag or serpentine shape.

24-37. (canceled)

38. An implantable stimulation arrangement, comprising:
an elongate stimulation assembly configured to be implanted in a recipient, wherein the elongate stimulation assembly comprises a plurality of electrodes;
a lead; and
an angular discontinuity disposed between a proximal end of the elongate stimulation assembly and a distal end of the lead, wherein the angular discontinuity comprises a pre-formed bend and an extension region that is non-parallel to an axis of a straight portion of a distal section of the elongate stimulation assembly within a body chamber of the recipient.

39. The implantable stimulation arrangement of claim 38, wherein the lead comprises a resiliently flexible body and a plurality of wires extending through the resiliently flexible body electrically connecting the plurality of electrodes to an implantable stimulator unit.

40. The implantable stimulation arrangement of claim 38, wherein the pre-formed bend comprises an angle in a range of approximately 45 degrees to approximately 120 degrees relative to the axis of the straight portion of the distal section of the elongate stimulation assembly within the body chamber of the recipient.

41. The implantable stimulation arrangement of claim 38, wherein the pre-formed bend comprises an angle of approximately 90 degrees relative to the axis of the straight portion of the distal section of the elongate stimulation assembly within the body chamber of the recipient.

42. The implantable stimulation arrangement of claim 38, wherein the pre-formed bend comprises an angle of between approximately 90 degrees and approximately 120 degrees relative to the axis of the straight portion of the distal section of the elongate stimulation assembly within the body chamber of the recipient.

43. The implantable stimulation arrangement of claim 38, wherein the pre-formed bend comprises an angle of between approximately 45 degrees and approximately 90 degrees relative to the axis of the straight portion of the distal section of the elongate stimulation assembly within the body chamber of the recipient.

44. The implantable stimulation arrangement of claim 38, wherein the extension region comprises an entire length of the lead.

45. The implantable stimulation arrangement of claim 38, wherein the proximal end of the elongate stimulation assembly has a diameter, and wherein the extension region has a length that is at least five times the diameter of the elongate stimulation assembly.

46. The implantable stimulation arrangement of claim 45, wherein the extension region has a length that is at least ten times the diameter of the elongate stimulation assembly.

47-53. (canceled)
